(19) United States
(12) Patent Application Publication
(10) Pub. No.: US 2015/0376701 A1
(43) Pub. Date: Dec. 31, 2015

(54) BIOMARKERS OF AUTOIMMUNE AND/OR CHRONIC DISEASES ASSOCIATED WITH JOINT INFLAMMATION

(71) Applicant: THE UNIVERSITY OF BIRMINGHAM, Birmingham (GB)

(72) Inventors: Dagmar Scheel-Toellner, Birmingham (GB); Lorraine Yeo, Brimingham (GB); Karim Raza, Birmingham (GB); Andrew Filer, Birmingham (GB); Christopher Buckley, Birmingham (GB)

(73) Assignee: The University of Birmingham, Brimingham (GB)

(21) Appl. No.: 14/764,671

(22) PCT Filed: Jan. 30, 2014
(86) PCT No.: PCT/GB2014/050256

§ 371 Date: Jul. 30, 2015

(30) Foreign Application Priority Data
Jan. 31, 2013 (GB) 1301708.2
Jun. 10, 2013 (GB) 1310282.7

(51) Int. Cl. C12Q 1/68; C07K 16/28; G01N 33/569

(52) U.S. Cl. CPC C12Q 1/6883; G01N 33/56972; C07K 16/2803; G01N 2800/102; G01N 2333/70535; G01N 2333/70575; C12Q 2600/158; C07K 2317/73

(57) ABSTRACT

The present invention relates to methods and uses of FcRL4 expression and optionally RANKL expressing B cells as biomarkers and therapeutic target for treatment of an autoimmune and/or chronic diseases associated with joint inflammation, in particular Rheumatoid Arthritis (RA).

The present invention relates to methods and uses of FcRL4 expression and/optionally RANKL expressing B cells as biomarkers and therapeutic target for treatment of autoimmune diseases associated with joint inflammation, in particular Rheumatoid Arthritis (RA).

### INTRODUCTION

Rheumatoid arthritis (RA) is a chronic systemic disease characterised by inflammation and destruction of the joints. The importance of the B cell lineage in RA pathogenesis is highlighted by the clinical effectiveness of B cell-targeting therapies. These include Rituximab (trade names Rituxan and MabThera) which is a chimaeric therapeutic monoclonal antibody against the protein CD20 and which depletes most CD20-expressing B cells. Ocrelizumab and Ofatumumab are humanised and completely human antibodies that also target CD20. There are also other B cell targeting antibodies under development including the antibody epratuzumab (which targets anti-CD22, another B cell marker). Potential mechanisms by which B cells drive disease processes in RA include their ability to produce autoantibody, act as antigen-presenting cells, or secrete cytokines; however, the exact role of B cells in RA remains unclear.

Our recent findings suggest a pro-inflammatory and destructive role for B cells via production of cytokines including RANKL, TNF-α and IL-6 (Yeo, Toellner et al. 2011). The cytokine RANKL (being the Ligand for RANK (Receptor Activator of Nuclear factor Kappa-B), is also known as tumour necrosis factor ligand superfamily member 11 (TNFSF11), TNF-related activation-induced cytokine (TRANCE), CD254, osteoprotegerin ligand (OPGL), and osteoclast differentiation factor (ODF). RANKL, in humans, is encoded by the TNFSF11 gene. Its main role is thought to be in bone resorption and development of lymphatic tissue (Kong, Yoshida et al. 1999).

Surprisingly, we have found a pro-inflammatory subset of memory B cells, FCRL4+ B cells, that also produces RANKL in the rheumatoid synovium. In particular, we have identified a B cell subset present in the RA synovium which has not previously been described in RA or any other chronic inflammatory arthritis. This B cell subset has been found in the peripheral blood of patients with malaria, cryoglobulinaemia and Hepatitis, but not in the joint. This memory B cell subset is characterised by expression of FcRL4. FcRL4 was previously known as an inhibitory receptor capable of aborting B cell receptor-mediating signalling and proliferation (Ehrhardt, Davis et al. 2003; Kardava, Moir et al. 2011). Alternative names for FcRL4 include IRTA1, (immunoglobulin superfamily receptor translocation-associated CD307d, FCRH4 and IGFP2. FcRL4 is therefore now thought to play an important role in the control of immune responses through regulation of B cell activation and differentiation. However, what is particularly useful is that FcRL4 expression and/optionally RANKL expression by B cells are prognostic for autoimmune diseases associated with joint inflammation, in particular RA, or can be used to stratify patients in relation to said conditions. FcRL4 expression and/optionally RANKL expression are also useful in targeting administration of B cell targeting therapies, such as Rituximab and the other antibodies described herein, to those patients responsive to the antibody.

We have also made a number of important discoveries with commercial relevance based on expression patterns of FcRL4 mRNA. One of these is that FcRL4 mRNA is not expressed in uninflamed synovial tissue but can be detected in synovium and peripheral blood mononuclear cells from patients with RA. This shows that FcRL4 represents an attractive therapeutic target and can be used as biomarker for RA, as its expression in the synovium is restricted to the disease state.

Furthermore, FcRL4 mRNA expression in synovial tissue is also important in predicting a patient's response to rituximab. Finally, FcRL4 mRNA expression can be detected in peripheral blood mononuclear cells of a proportion of RA patients. FcRL4 RNA may be detected by methods such as real-time PCR.

### SUMMARY OF THE INVENTION

The following are therefore useful as biomarkers for progression to autoimmune and/or chronic disease associated with joint inflammation: the presence of FcRL4 as RNA or as a B cell marker (FcRL4+ cells) in the synovium or blood; and/or the presence of RANKL-expressing B cells in the synovium or blood. Presence in the synovium may typically be determined by sampling the synovial tissue or fluid.

Thus, in a first aspect, the invention provides use of FcRL4 RNA, FcRL4-expressing B cells and/optionally RANKL-expressing B cells, in a blood sample or a synovial tissue or fluid sample, as a prognostic marker for, autoimmune and/or chronic disease associated with joint inflammation.

Autoimmune and/or chronic disease associated with joint inflammation may, in particular, be RA (Rheumatoid Arthritis) ankylosing spondylitis and psoriatic arthritis. These biomarkers are particularly useful for distinguishing between chronic disease and resolving disease, the presence of one or more of these biomarkers being indicative of chronic disease (which includes the likelihood of progression thereto), rather than resolving disease. The B cells may express FcRL4 (for instance through presentation of this receptor on their cell surface). They may also express RANKL and be ultimately transported or secreted into the surrounding synovial tissue or fluid).

Also provided is a method of determining the likelihood of a patient developing autoimmune and/or chronic disease associated with joint inflammation, in particular RA (Rheumatoid Arthritis) ankylosing spondylitis and psoriatic arthritis, the method comprising determining the presence of FcRL4 RNA, FcRL4-expressing B cells and/optionally RANKL-expressing B cells, in a blood sample or a synovial tissue or fluid sample, from said patient.

Further provided is a method for identifying a patient likely to develop an autoimmune and/or chronic disease associated with joint inflammation, in particular RA, ankylosing spondylitis and psoriatic arthritis, comprising:
a) determining the presence of FcRL4 RNA, FcRL4-expressing B cells and/optionally RANKL-expressing B cells, in a blood sample or a synovial tissue or fluid sample, from the patient;
b) comparing the determined level of said biomarker(s) with a reference level; and
c) identifying the patient based on the comparison of b).

Also provided is a screening method for identifying, within a population, one or more patients likely to develop an autoimmune and/or chronic disease associated with joint inflammation, in particular RA, ankylosing spondylitis and psoriatic arthritis, comprising:
a) determining the presence of FcRL4 RNA, FcRL4-expressing B cells and/optionally RANKL-expressing B cells, in a blood sample or a synovial tissue or fluid sample, from each patient;
b) comparing the determined level of said biomarker(s) with a reference level; and
c) identifying patients based on the comparison of b).

Provided is also a method of determining the likelihood of a patient developing an autoimmune and/or chronic disease associated with joint inflammation, in particular RA ankylosing spondylitis and psoriatic arthritis, comprising:
a) optionally, obtaining a blood sample or a synovial tissue or fluid sample from a patient;
b) contacting the sample with a biopharmaceutical specific for FcRL4, such as an anti-FcRL4 antibody, and/optionally a biopharmaceutical specific for RANKL, such as an anti-RANKL antibody;
c) comparing the binding of the biopharmaceutical or biopharmaceuticals to a reference level; and
d) determining that the patient is likely to develop the disease based upon the increased binding of said biopharmaceutical or biopharmaceuticals.

Also provided is a method of determining the likelihood of a patient developing an autoimmune and/or chronic disease associated with joint inflammation, in particular RA ankylosing spondylitis and psoriatic arthritis, comprising:
a) optionally, obtaining a blood sample, in particular a peripheral blood sample, or a synovial tissue or fluid sample from a patient;
b) amplifying polynucleotides encoding FcRL4, in particular FcRL4 RNA, in the sample;
c) the presence of said polynucleotides encoding FcRL4 being indicative that the patient is likely to develop the disease.

The amplifying step may include quantification of the polynucleotides encoding FcRL4. Suitable methods for amplifying polynucleotides encoding FcRL4 include PCR and many known variants thereof, such as qPCR (also known as real time PCR).

In general, in any of the present methods, determining the presence of FcRL4-expressing B cells and/optionally RANKL-expressing B cells may comprise:
contacting the sample with a biopharmaceutical specific for FcRL4, such as an anti-FcRL4 antibody, and/optionally a biopharmaceutical specific for RANKL, such as an anti-RANKL antibody;
comparing the binding of the biopharmaceutical or biopharmaceuticals to a reference level; and
determining that the patient is likely to develop the disease based upon the increased binding of said biopharmaceutical or biopharmaceuticals.

Alternatively, in general, in any of the present methods, determining the presence of FcRL4 RNA may comprise:
amplifying polynucleotides encoding FcRL4;
the presence of said polynucleotides encoding FcRL4 being indicative that the patient is likely to develop the disease.

As mentioned above, the amplification step preferably includes PCR. Optionally, the method includes a quantification of the amplified polynucleotides, so that a preferred step is to amplify and quantify the polynucleotides encoding FcRL4. This may be achieved by ‘quantitative’ or ‘real-time’ PCR (generally referred to as qPCR) or Real-time Reverse Transcription PCR (generally referred to as qRT-PCR), for instance. qPCR may include: use of non-specific fluorescent dyes that intercalate with any double-stranded DNA; or sequence-specific DNA probes consisting of oligonucleotides that are labelled with a fluorescent reporter which permits detection only after hybridization of the probe with its complementary DNA target. Suitable probes for FcRL4 RNA can be designed using the known sequence of the FcRL4 coding sequence. For example, the amino acid sequence of the FcRL4 protein is provided herein and from that a DNA sequence can be predicted and suitable probes designed to hybridise thereto. The DNA coding sequence is also known and available on the internet and databases such as EMBL database. Reference to FcRL4 RNA is made herein and it will be understood that this is principally in respect of transcript (mRNA), but that this can be in processed on unprocessed form.

Another use of anti-FcRL4 and/optionally anti-RANKL antibodies (biopharmaceuticals) is to provide an in vitro diagnostic use. Thus, the invention provides in vitro use of a biopharmaceutical specific for FcRL4, such as an anti-FcRL4 antibody, and/optionally a biopharmaceutical specific for RANKL, such as an anti-RANKL antibody for identifying a patient likely to develop an autoimmune and/or chronic disease associated with joint inflammation, in particular RA, ankylosing spondylitis and psoriatic arthritis, wherein the identified patient displays, in a blood sample or a synovial tissue or fluid sample, a level of FcRL4 and/or RANKL protein which is greater than a reference level.

Also provided is a method of treating a patient with autoimmune and/or chronic disease associated with joint inflammation, in particular RA, ankylosing spondylitis and psoriatic arthritis, comprising:
a) determining, as described herein, the likelihood of a patient having or developing said disease; and
b) administering a suitable treatment (such as a B cell depleting therapy, e.g. an anti-CD20 antibody (e.g Rituximab) or an FcRL4 targeting therapy such as an anti-FcRL4 antibody) to a patient determined to have said condition or to a patient likely to develop said condition.

Treatment for RA and other identified diseases such as the use of an anti-CD20 antibody, such as Rituximab (also known as Rituxan and MabThera). Suitable treatments, particularly for RA, ankylosing spondylitis and psoriatic arthritis, are well-known and may include administration of any of the biopharmaceuticals (including antibodies) described herein.

The following applies to all uses and methods described herein unless otherwise apparent.

Resolving disease may be in fact a condition resulting from an injury. For instance, generalised inflammation caused by an injury may in time subside or heal without intervention or through treatment with treated with anti-inflammatory drugs such as NSAIDs or steroids.

The terms ‘marker’ and biomarker may be used interchangeably. Typically, an increase or a noticeable presence of one or both of the biomarkers is indicative of a likelihood of developing an autoimmune and/or chronic disease associated with joint inflammation, in particular RA, ankylosing spondylitis and psoriatic arthritis. Alternatively, a decrease or a noticeable absence of one or both of the biomarkers is indicative of a likely not developing an autoimmune and/or chronic disease associated with joint inflammation, in particular RA, ankylosing spondylitis and psoriatic arthritis, i.e. that the joint inflammation or pain is resolving disease. The reference levels are optionally the same as those provided elsewhere herein.

Optionally, the biomarker may be a RANKL-expressing B cell (RANKL+ B cell). As FcRL4 and RANKL have been shown by the invention to be linked, the prognostic biomarkers may be FcRL4, FcRL4+ cells optionally RANKL-expressing B cells. The presence of RANKL in synovial fluid is known and so is excluded, unless associated with expression from B cells. These biomarkers may be considered separately (FcRL4 alone, FcRL4+ cells alone or B cells which express FcRL4 and RANKL optionally) in any combination (FcRL4 and FcRL4+ cells; FcRL4 and RANKL-expressing B cells; or FcRL4+ cells and RANKL-expressing B cells).

The biomarker is FcRL4. This may be detected in amino acid (peptide or protein) form, such as this biomarker expressed on the cell surface (a FcRL4+ cell), in particular of a B cell (a FcRL4+ B cell). Alternatively, FcRL4 may be detected in transcript (e.g. RNA, especially mRNA) form, for instance via PCR methods including qPCR. Detection of RNA polynucleotides encoding FcRL4 is preferred. These are advantageous as we have shown that they are present in not only synovial tissue or fluid, but also in peripheral blood. The presence of such FcRL4 RNA is indicative of responsiveness of the patient (from whom the sample originates) is likely to be responsive to B cell depleting therapies, including anti-CD20 biopharmaceuticals, such as anti-CD20 antibodies, in particular Rituximab. As mentioned herein, suitable detection methods include amplification and quantification of the RNA polynucleotides encoding FcRL4, including qPCR. The RNA polynucleotides encoding FcRL4 may be the full transcript or just a fragment provided that the fragment is sufficiently long to provide a reasonable degree of certainty that its presence is indicative of the presence of FcRL4 (i.e. to avoid a false-positive). Polynucleotides of around 15, 20 or preferably 25 nucleotides in length should be sufficient for this purpose.

Sampling the peripheral blood is advantageous as it is easier and less invasive than sampling synovial fluid or tissue. It is also potentially less painful as the sampling can occur away from what may be an already painful joint.

Data from gene expression databases suggests that FcRL4 expression is highly restricted to this (a FcRL4-expressing) B cell subset. Gene expression databases show that, across 32 different tissue types from healthy individuals, this gene is only otherwise expressed in the tonsil and at much lower levels in spleen and salivary glands.

Optionally, therefore, the sample is blood, for instance the peripheral blood.

The sample may also be synovial fluid or synovial tissue (for instance biopsied synovial tissue or a cell preparation). Peripheral blood and/or synovial fluid cell preparations are preferred. Again, quantitative PCR (qPCR) is particularly preferred for determining the presence of FcRL4 expression or FcRL4+ cells, especially from peripheral blood and/or synovial fluid samples.

It is also envisaged that RANKL mRNA can be detected, for instance by amplification methods such as qPCR. This may require isolating the B cells first. An alternative would be to use a method such as in-situ hybridisation for detecting RANKL mRNA.

Indeed, it is also preferred that in-situ hybridisation may also be used to detect FcRL4 mRNA.

The autoimmune and/or chronic disease associated with joint inflammation, may be lupus. Optionally, the disease may be an autoimmune and/or chronic diseases associated with joint inflammation, which is an arthritis or sclerosis. The arthritis may optionally be psoriatic arthritis, ankylosing spondylitis or RA, which is preferred. Forms of arthritis not associated with autoimmune and/or chronic disease are excluded. The present conditions are grouped under the definition of arthritide, autoimmune and/or chronic disease associated with joint inflammation. This is predicated on the widely-held view that these conditions are in fact, autoimmune and/or chronic diseases. In may be that ultimately a condition such as RA is deemed not to be an autoimmune and/or chronic disease, but it will be appreciated that at present it is considered to be an autoimmune and/or chronic disease and thus falls within the definition of, autoimmune and/or chronic disease associated with joint inflammation. If necessary, the present conditions could be defined as autoimmune and/or chronic disease associated with joint inflammation and RA, ankylosing spondylitis and psoriatic arthritis, for instance.

The autoimmune and/or chronic disease associated with joint inflammation may even be ankylosing spondylitis, as a gene single nucleotide polymorphism in the FCRL4 gene has been associated with ankylosing spondylitis in the Chinese Han population (Association of FcRL4 polymorphisms on disease susceptibly and severity of ankylosing spondylitis in Chinese Han population: Zou D, Pan F). It should be noted that the association was not in the context of B cells. Reference is made to FcRL3 (not 4), but the link with RA is shown to be groundless. In some embodiments, however, ankylosing spondylitis is excluded.

The FcRL4 and/optionally RANKL determined may be expressed protein or a fragment thereof present in the sample. Optionally, the FcRL4 and/optionally RANKL may be determined by the presence of FcRL4 and/optionally RANKL on the cell surface, in particular of B cells. As such, a preferred option is that the FcRL4 and/optionally RANKL determined may be via detection of FcRL4+ and/optionally RANKL+ B cells.

| SEQ ID NO: 1 Amino acid sequence of FCRL4 from UniProtKB/Swiss-Prot: Q96PJ5.1: | |
|---|---|
| 1 | MLLWASLLAF APVCGQSAAA HKPVISVHPP WTTFFKGERV TLTCNGFQFY ATEKTTWYHR |
| 61 | HYWGEKLTLT PGNTLEVRES GLYRCQARGS PRSNPVRLLF SSDSLILQAP YSVFEGDTLV |
| 121 | LRCHRRRKEK LTAVKYTWNG NILSISNKSW DLLIPQASSN NNGNYRCIGY GDENDVFRSN |
| 181 | FKIIKIQELF PHPELKATDS QPTEGNSVNL SCETQLPPER SDTPLHFNFF RDGEVILSDW |
| 241 | STYPELQLPT VWRENSGSYW CGAETVRGNI HKHSPSLQIH VQRIPVSGVL LETQPSGGQA |
| 301 | VEGEMLVLVC SVAEGTGDTT FSWHREDMQE SLGRKTQRSL RAELELPAIR QSHAGGYYCT |
| 361 | ADNSYGPVQS MVLNVTVRET PGNRDGLVAA GATGGLLSAL LLAVALLFHC WRRRKSGVGF |
| 421 | LGDETRLPPA PGPGESSHSI CPAQVELQSL YVDVHPKKGD LVYSEIQTTQ LGEEEEANTS |
| 481 | RTLLEDKDVS VVYSEVKTQH PDNSAGKISS KDEES |
| SEQ ID NO: 2 Amino acid sequence of RANKL, also known as TNFSF11, from | |
| UniProtKB/Swiss-Prot: O14788.1 | |
| 1 | MRRASRDYTK YLRGSEEMGG GPGAPHEGPL HAPPPPAPHQ PPAASRSMFV ALLGLGLGQV |
| 61 | VCSVALFFYF RAQMDPNRIS EDGTHCIYRI LRLHENADFQ DTTLESQDTK LIPDSCRRIK |
| 121 | QAFQGAVQKE LQHIVGSQHI RAEKAMVDGS WLDLAKRSKL EAQPFAHLTI NATDIPSGSH |
| 181 | KVSLSSWYHD RGWAKISNMT FSNGKLIVNQ DGFYYLYANI CFRHHETSGD LATEYLQLMV |
| 241 | YVTKTSIKIP SSHTLMKGGS TKYWSGNSEF HFYSINVGGF FKLRSGEEIS IEVSNPSLLD |
| 301 | PDQDATYFGA FKVRDID |

The level (of the, or each biomarker) determined may be by expression of said biomarker. This may be the protein or polynucleotide encoding it. The sequences of the proteins are given above and polynucleotides encoding them can be easily envisaged. The polynucleotide encoding the protein biomarkers may be DNA, but is most typically RNA, in particular messenger RNA (mRNA). Determining the level of FcRL4 RNA may be achieved by known methods, such as qPCR.

The levels of protein and mRNA expression can be determined independently (i.e. FcRL4 or RANKL). When levels of both biomarkers are determined (i.e. FcRL4 and RANKL) then, typically, either protein levels of both or RNA levels are looked at. It is considered to be advantageous to combine the determination of the levels of both biomarkers.

As FcRL4 is a cell bound protein determining the level of protein expression is typically achieved by measuring the prevalence of the protein form of the biomarker on the cell surface. This may be achieved by known methods, for example, staining on tissue sections with specific antibodies. Bound antibody would be made visible with fluorescent secondary antibodies and analysed by fluorescence microscopy. Alternatively antibody binding could be made detected by enzyme-labelled secondary antibodies and enzymatic generation of a coloured precipitate, which would be observed using light microscopy.

In cell suspensions from peripheral blood or synovial fluid or dissociated synovial tissue, FcRL4 can be labelled by specific antibodies either directly conjugated with fluorescent molecules or with fluorescent dye-conjugated secondary antibodies which can be analysed and quantified in a flow cytometer, for instance.

Cell bound RANKL can be detected by the same methods described above for FcRL4, however it can also be released from the cells. Released RANKL can be detected by Enzyme-linked immunosorbent assays (ELISA). Intracellular RANKL can also be detected by the above methods i.e. fluorescence microscopy, light microscopy or flow cytometry, RANKL is a cytokine and so is expressed and then may be retained on the B cell surface or released by the B cells. As such, RANKL-expressing B cells includes reference to B cells where RANKL has been retained on the cell surface or released therefrom.

Patients not likely to develop the autoimmune and/or chronic diseases associated with joint inflammation, have other conditions, such as resolving disease. These patients may present with joint inflammation, but ultimately do have an autoimmune and/or chronic disease associated with joint inflammation. ‘Resolving disease’ is used herein to cover a range of possible conditions, other than the, autoimmune and/or chronic diseases associated with joint inflammation, with may be causing the pain or inflammation in the joint or joints of the patients. Principal among these conditions is inflammation that can be treated effectively with NSAIDs for instance.

The patient or patients identified may be those that are likely to develop autoimmune and/or chronic diseases associated with joint inflammation, such as RA, ankylosing spondylitis and psoriatic arthritis, or they may be those that are likely to develop resolving disease (which as mentioned above includes other conditions causing the symptoms). The present uses and methods therefore allow patients to be stratified according to their clinical outcome or likely clinical outcome.

For the sake of simplicity, the present specification will now refer to RA, but it will be appreciated that this is in fact reference to autoimmune and/or chronic diseases associated with joint inflammation, of which RA is an example. Furthermore, where reference is made throughout this specification to a patient who is “likely to develop RA,” it will be appreciated that this includes a patient that has RA, will develop RA, or is at least likely to go on to develop RA or any of the other identified conditions described herein. The same holds true, mutatis mutandis for resolving disease, so that a patient who is “likely not to develop RA” or “is likely to develop resolving disease,” “will develop resolving disease” or “has resolving disease” can be used interchangeably. Furthermore, it will also be appreciated that term “develop” may include “diagnose” so that a patient that is likely to develop RA may be one that is later diagnosed with RA according to the appropriate criteria. Here, the terms ‘RA’ and ‘early RA’ are also used interchangeably.

FcRL4 expression, being both FcRL4 protein expression on B cells and/or FcRL4 RNA (in the synovial tissue of fluid or peripheral blood), may distinguish between chronic and resolving disease or between RA and other chronic and resolving conditions. Both possibilities are useful. Responsiveness to biopharmaceuticals such as anti-CD-20 antibodies including Rituximab can also be indicated by the presence of FcRL4.

The joint inflammation may, therefore, simply be temporarily inflamed. Those patients stratified as having RA may have chronic RA or an early form thereof that goes on to develop into chronic RA. Typically, the patients satisfy the ACR 1987 or the ACR/EULAR 2010 criteria. Those patients stratified as having RA are actually patients in whose samples the presence of FcRL4 and/optionally RANKL-expressing B cells is determined.

Thus, these uses and methods can help to differentiate between patients likely to have a temporarily inflamed joint, or joints, i.e. with resolving disease and patients likely to develop RA. This stratification promotes rapid and effective treatment, saving time and money.

The reference level may not even be required. It may be enough merely that FcRL4 RNA, FcRL4-expressing B cells and/optionally RANKL-expressing B cells are found to be present in the sample. Typically, the patients are sampled within the first 3 months of symptom onset of the inflammation or its associated pain.

The determination of the presence of the biomarker(s) in a sample may equally be determined from said sample. A prognostic marker is a marker that indicates a certain prognosis i.e. a likelihood that the patient at issue will develop the autoimmune and/or chronic diseases associated with joint inflammation, particularly RA. This may assist with the diagnosis of the condition, by being one of a number of factors considered to provide the diagnosis, for instance that the patient has RA.

The increased binding in step d) is typically due to an increase in the proliferation of cells carrying the biomarker(s) or alternatively a survival advantage of these cells or increased migration of the cells carrying the biomarker into the affected tissue.

Examples of particular antibodies that recognise (i.e. are specific for epitopes on) FcRL4 and/optionally RANKL are or may be those described below. Antibodies to FcRL4 include mouse anti-human CD307d (Biolegend), rabbit anti-human IRTA1 (AbCam), goat anti-human FcRH4 (Santa Cruz Biotechnology), mouse anti-human FCRL4 (R&D Systems), goat anti-human FCRL4 (R&D Systems), rabbit anti-human IRTA1 (Thermo Scientific Pierce Antibodies), rabbit anti-human FCRL4 (Aviva Systems Biology) rabbit anti-human FCRL4 (Abnova Corporation), rabbit anti-human FCRL4 (Abgent), rabbit anti-human FCRL4 (Sigma Aldrich), and rabbit anti-human IRTA1 (Novus Biologicals). Antibodies to RANKL include mouse anti-human RANKL (Santa Cruz Biotechnology), goat anti-human RANKL (SantaCruz Biotechnology), rabbit anti-human RANKL (Santa Cruz Biotechnology), mouse anti-human CD254 (eBioscience), rat anti-human RANKL (BD Bioscience), rabbit anti-human RANKL (Novus Biologicals), rat anti-human RANKL (Novus Biologicals), and mouse anti-human RANKL (Merck Millipore).

The antibody may be human or humanised versions of any of the antibodies described herein. If that reference antibody is not human or humanised, then a humanised or human version thereof is preferred. The generation of humanised or human antibody from, for example, murine antibody is generally well known in the art. Any form of antibody is envisaged, such as domain antibodies, single chain variable region fragments or IgA/B/C/D/E/F and especially IgG (any of subtypes 1, 2 3 or 4). Indeed, where reference is made herein to an antibody, it will be appreciated that this includes biopharmaceuticals. These biopharmaceuticals may include humanised antibodies, domains and fragments of antibodies, chimeric antibodies, bi-specific antibodies, antibody-drug conjugates, non-immunoglobulin protein scaffolds including, but not restricted to adnexins, darpins, shark variable domains and non-protein domains including but not restricted to aptamers.

The negative prognosis for step d), i.e.: determining that the patient is likely not to develop an autoimmune and/or chronic disease associated with joint inflammation, in particular RA, is based upon the little or no binding of said antibody or antibodies due to the absence of FcRL4 and/optionally RANKL-expressing B cells.

It will be appreciated that step a) in certain methods described above may be deleted such that the first step is to contact a sample from a patient.

We have also shown that FcRL4+ B cells in RA synovial fluid are mostly switched memory B cells expressing high levels of CD20 and CD11c, and low levels of CD21. Accordingly, the target B cells may be defined in this manner.

The action of Rituximab and other B cell depleting therapies in rheumatoid arthritis is poorly understood. It kills the vast majority of peripheral blood B cells and also most of the tissue based B cells (Edwards and Cambridge 2001; Thurlings, Vos et al. 2008). Only at the latest stage of B cell differentiation, antibody producing plasma cells do not express the antigen CD20, and are not targeted directly by Rituximab. Only a small proportion of the B cells will at any given time contribute to inflammation in the patient. It is therefore unnecessary to eliminate most B cells. Thus, there are a number of side effects resulting from the broad-brush elimination of B cells by Rituximab. These include reduced effectiveness of vaccination in patients receiving this antibody (Oren, Mandelboim et al. 2008) and reduced antibody levels.

FcRL4 expression, typically in either amino acid or RNA form by B cells, especially from peripheral blood, synovial fluid or synovial tissue of RA patients, provides a useful biomarker for RA and also prediction of clinical response to Rituximab.

Thus, in a further aspect, the invention provides a method for identifying a patient likely to respond to a B cell depleting therapy, including an anti CD-20 biopharmaceutical such as Rituximab or an FcRL4 targeting therapy, comprising:
(i) determining the presence of FcRL4 RNA, FcRL4-expressing B cells and/optionally RANKL in a sample from the patient;
(ii) comparing the determined level of said biomarker(s) with a reference level; and
(iii) identifying the patient based on the comparison of ii).

Also provided is a screening method for identifying, within a population, one or more patients likely to respond to a B cell depleting therapy, including an anti-CD20 biopharmaceutical such as Rituximab or an FcRL4 targeting therapy, comprising:
(i) determining the presence of FcRL4 RNA, FcRL4-expressing B cells and/optionally RANKL-expressing B cells in a sample from each patient;
(ii) comparing the determined level of said biomarker(s) with a reference level; and
(iii) identifying patients based on the comparison of ii).

Also provided is a method of determining the likelihood of a patient responding to a B cell depleting therapy, including an anti CD20 biopharmaceutical such as Rituximab or an FcRL4 targeting therapy, comprising:
(i) obtaining a sample from a patient;
(ii) contacting the sample with an a biopharmaceutical specific for FcRL4, such as an anti-FcRL4 antibody, and/optionally a biopharmaceutical specific for RANKL, such as an anti-RANKL antibody;
(iii) comparing the binding of the biopharmaceutical or biopharmaceuticals to a reference level; and
(iv) determining that the patient is likely to develop the disease based upon the increased binding of said biopharmaceutical or biopharmaceuticals.

Another option based on the use of anti-FcRL4 and/optionally anti-RANKL antibodies (biopharmaceuticals) is for an in vitro diagnostic use. Thus, the invention provides in vitro use of a biopharmaceutical specific for FcRL4, such as an anti-FcRL4 antibody, and/optionally a biopharmaceutical specific for RANKL, such as an anti-RANKL antibody for identifying a patient likely to respond to Rituximab, wherein the identified patient displays in a sample a level of FcRL4 and/optionally RANKL protein which is greater than a reference level.

Typically, an increase or a noticeable presence of FcRL4 and/optionally RANKL or FcRL4+ and/optionally RANKL+ B cells is indicative of a likely positive response to and an anti-CD20 biopharmaceutical such as Rituximab. Alternatively, a decrease or a noticeable absence of FcRL4 and/or RANKL or FcRL4+ and/optionally RANKL+ B cells is indicative of a likely negative response to an anti-CD20 biopharmaceutical such as Rituximab or to an FcRL4 targeting therapy as described therein. The reference levels are optionally the same as those provided elsewhere herein.

Optionally, the prognosis of increased likelihood of developing an, autoimmune and/or chronic disease associated with joint inflammation, in particular RA, is twinned with an increased likelihood that the same patient is responsive to Rituximab.

Although reference has been made to Rituximab, other B cell-targeting antibodies, including those described herein, can be interchanged with Rituximab. Alternatives to Rituximab are envisaged, such as alternative Antibody recognising CD20 (for example Ofatumumab and Ocrelizumab) or CD22 (for example Epratuzumab) or small molecules or biologics having an equivalent function to Rituximab. Equally, the use of anti-FcRL4 antibodies may be replaced by amplifications of polynucleotides encoding FcRL4 to thereby detect the FcRL4 transcript, for instance via PCR and especially qPCR, as described elsewhere.

The invention also provides FcRL4 as a therapeutic target for treating an, autoimmune and/or chronic disease associated with joint inflammation, in particular RA. We have identified a subset of B cells not previously been found at sites of chronic joint inflammation which is characterised by expression of FcRL4 and is capable of producing the bone-destructive and pro-inflammatory cytokines RANKL and TNF-α. This is first discussed in Example 1 and further weight to this is provided by Example 4.

This is an important observation, as TNF-α is a main driver of inflammation. It is a well validated clinical target in the treatment of rheumatoid arthritis and other, autoimmune and/or chronic inflammatory conditions. Specific, direct targeting of FcRL4 expressing B cells would allow removal of the pro-inflammatory B cells while sparing the vast majority of B cells. In patients undergoing B cell depleting therapies, immunoglobulin levels are compromised in the long term and response to vaccination is limited. If the pro-inflammatory B cells are directly targeted the majority of the B cell response will remain intact.

It will be appreciated that the reference herein to an antibody also covers biopharmaceuticals in general as herein described. FcRL4 therefore represents an attractive therapeutic target for depletion or removal of pro-inflammatory B cells as its expression is specific for B cells and its surface expression may readily facilitate effective blocking by antibodies/biopharmaceuticals. Use of an anti-FcRL4 antibody or biopharmaceutical, as described herein, is provided, for the depletion or removal of pro inflammatory B cells in the synovium or a sample. Methods for the treatment or prophylaxis of the conditions mentioned herein is also provided, comprising administering an anti-FcRL4 antibody, as described herein, to thereby deplete or remove pro inflammatory B cells in the synovium or a sample.

This targeting may involve inhibition of B cell function and activation, direct killing of the FcRL4 expressing B cells by binding of the antibody, or binding of toxins to anti-FcRL4 antibodies to target these toxins to the FcRL4 expressing B cells. Consequences of the removal or inhibition of these cells will lead to the reduction of local levels of proinflammatory cytokines and downregulation of inflammation. Beyond antibody production and cytokine expression a third function of B cells in the synovium is the presentation of antigens to T cells. Tonsillar and synovial FcRL4 positive B cells express higher levels of costimulatory molecules CD80 and CD86 when compared to FcRL4 negative B cells. (example 4 FIG. 8) These cells therefore are likely to be more active stimulators of T cells. Targeting of FcRL4 expressing B cells therefore is potentially also reducing T cell activation locally or in the draining lymph nodes.

Provided therefore, is the use of FcRL4—expressing B cells as therapeutic targets for the treatment of autoimmune and/or chronic disease associated with joint inflammation. This may be by administering an agent specific for FcRL4 to thereby target and/or remove B cells expressing FcRL4. In other words, the B cells may be targeted by an antibody specific for FcRL4. The use may be as a method for the treatment or prophylaxis of autoimmune and/or chronic disease associated with joint inflammation, comprising administering an agent specific for FcRL4 to thereby target and/or remove B cells expressing FcRL4. The agent specific for FcRL4 may be an antibody (as described herein).

Thus, provided is an antibody, specific for FcRL4, which is a humanised or human the anti-FcRL4 antibody. For instance, this may be a humanised or human version of the anti-FcRL4 antibodies mentioned herein. Also provided is a method of treating an autoimmune and/or chronic disease associated with joint inflammation, in particular RA, ankylosing spondylitis and psoriatic arthritis, comprising administering an agent, such as a small molecule inhibitor or antibody specific for FcRL4, to a patient. The antibody may be a domain antibody; a single (heavy or light) chain antibody; or an antibody comprising at least one light chain and at least one heavy chain, preferably 2 of each.

Functional work done on FcRL4 suggests that it is an inhibitory receptor which if activated can dampen down B cell receptor signalling. There is therefore a range of methods to target FcRL4 expressing cells which could be used: antibodies or small molecule compounds could mimic the interaction with the ligand and, rather than killing the B cells, suppress their activation. Furthermore, antibodies may directly kill the target cells by mechanisms similar to the action of Rituximab. Thirdly, FcRL4 specific antibodies could be coupled to drugs that could specifically kill the FcRL4 expressing target cells.

Therefore, FcRL4 may also be a useful therapeutic target. For instance, B cells expressing FcRL4 in the blood or synovium, which have a role in the present autoimmune and/or chronic disease, in particular RA, may be targeted. Indeed, although Rituximab also targets B cells, we are proposing to target B cells in a novel way using antibodies against FcRL4. Although antibodies against FcRL4 are known, they have not been used to target B cells associated with the present, autoimmune and/or chronic disease, in particular RA, ankylosing spondylitis and psoriatic arthritis. The targeting of these B cells via FcRL4 may include the removal or destruction of said B cells. Thus, in a further aspect, the invention provides a method for the treatment or prophylaxis of, autoimmune and/or chronic disease associated with joint inflammation, in particular RA, ankylosing spondylitis and psoriatic arthritis, comprising administering an antibody specific for FcRL4+ B cells. The anti-FcRL4 antibody may be any of those mentioned herein and if that antibody is not human or humanised, then a humanised or human version thereof, as mentioned above.

As an additional alternative to antibody based assays, we have advantageously detected gene expression for FCRL4 by a quantitative PCR protocol in peripheral blood and synovial fluid cell preparations. For instance this means that the attending clinician may only be required to transfer peripheral blood, synovial fluid or synovial needle biopsies into a commercially available container with fixative and send it to the lab for detection of FCRL4 mRNA expression using widely available generic technology.

The discussion above regarding samples and so forth applies equally across all methods and uses of FcRL4 and/optionally RANKL described herein unless otherwise apparent.

It will be appreciated that reference to RNA may refer to any type of RNA, although messenger RNA (mRNA) is preferred. The RNA may be in its processed or unprocessed form.

It will also be appreciated that reference to “FcRL4 and/optionally RANKL-expressing B cells” typically means FcRL4-expressing B cells; and optionally RANKL-expressing B cells. In some instances, said reference to FcRL4 may also encompass FcRL4 RNA, so that any of the following FcRL4 RNA; FcRL4-expressing B cells; and/optionally RANKL-expressing B cells are envisaged and in any combination. Preferably, one would look for either or both of the B cells (FcRL4-expressing B cells and/or RANKL-expressing B cells), or just at the FcRL4 RNA. A mixture of tests is also useful, so determining the presence of FcRL4 RNA with RANKL-expressing B cells is preferred. Alternatively, it may also be useful to concentrate only FcRL4, in which case the presence of either or both of FcRL4 RNA and FcRL4-expressing B cells is envisaged. Determining the presence of FcRL4 RNA is preferred in some embodiments, whilst determining the presence FcRL4-expressing B cells is preferred in others. In some embodiments, it is preferred to determine the presence of RANKL-expressing B cells.

### BRIEF DESCRIPTION OF THE FIGURES

FIG. 1—FcRL4 expression by CD19+ synovial fluid B cells

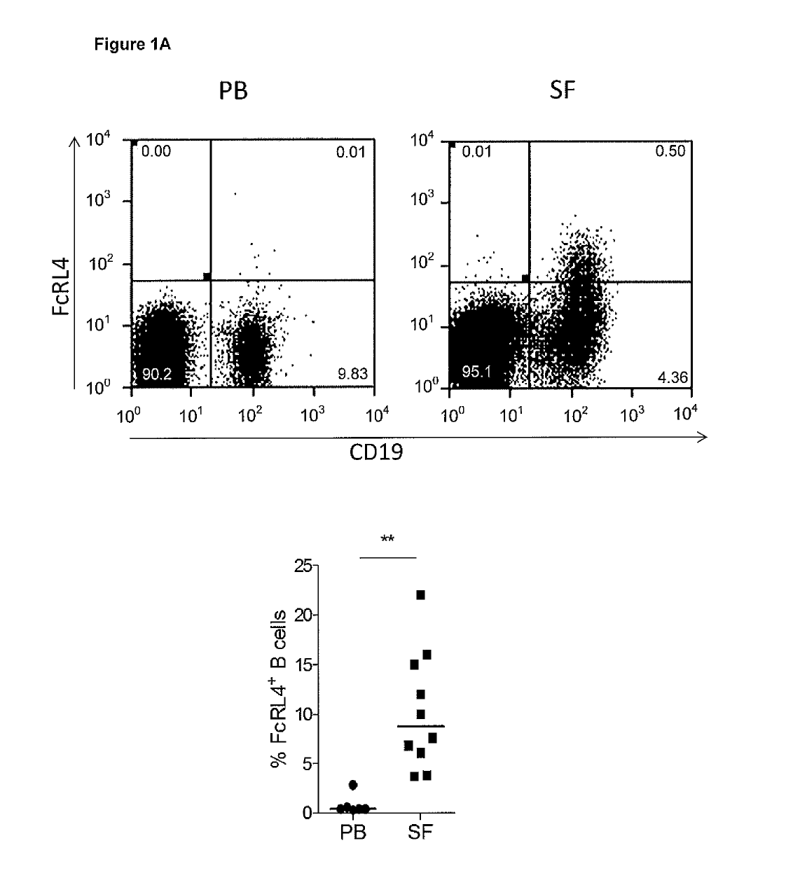

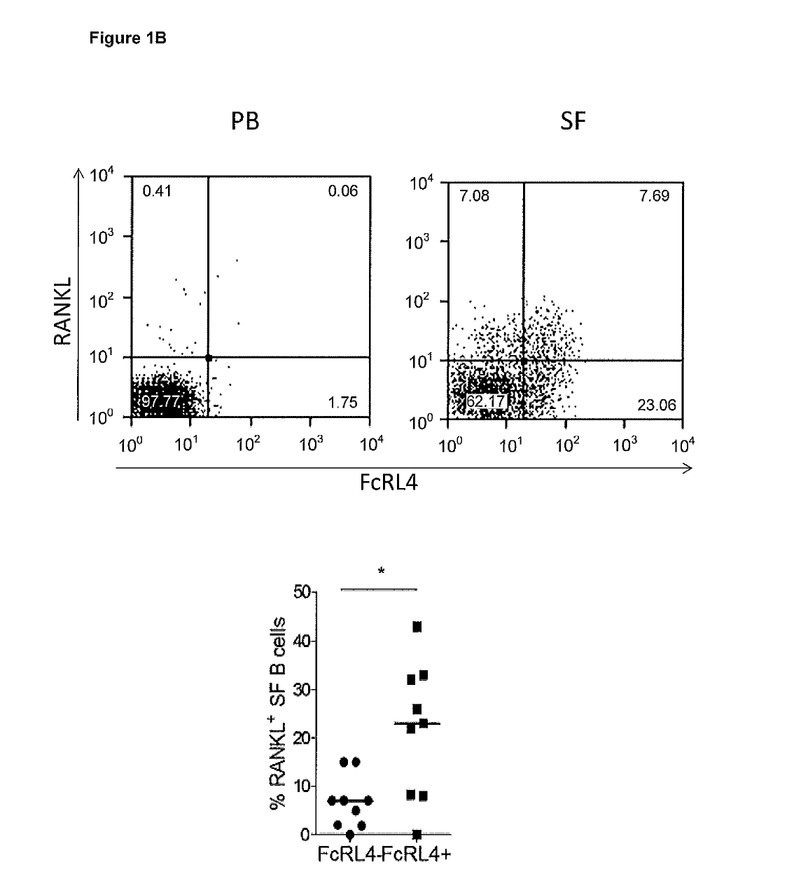

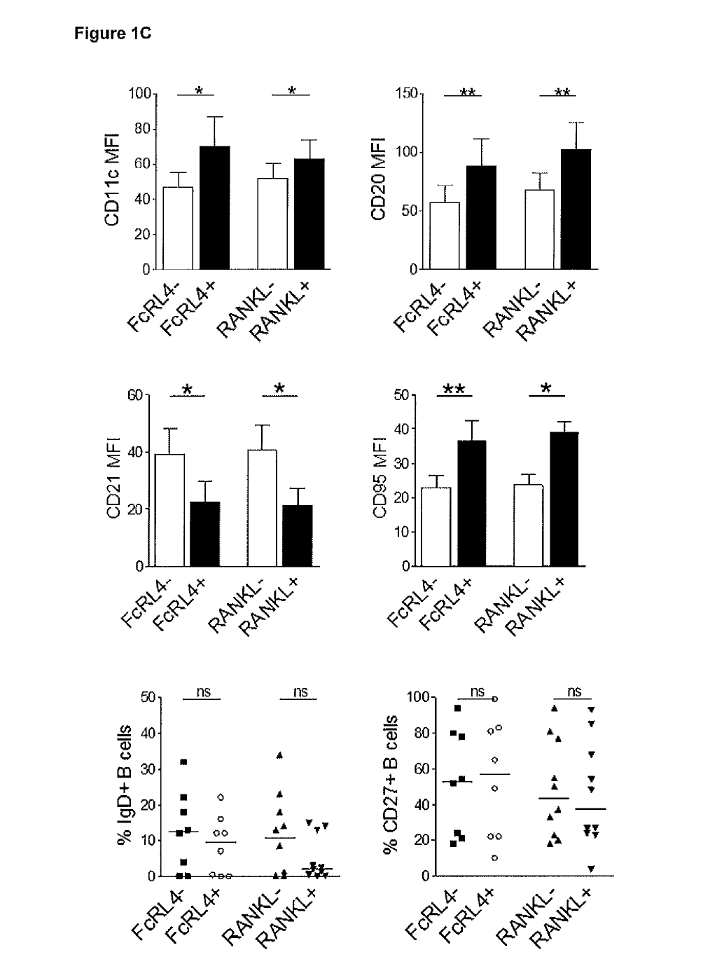

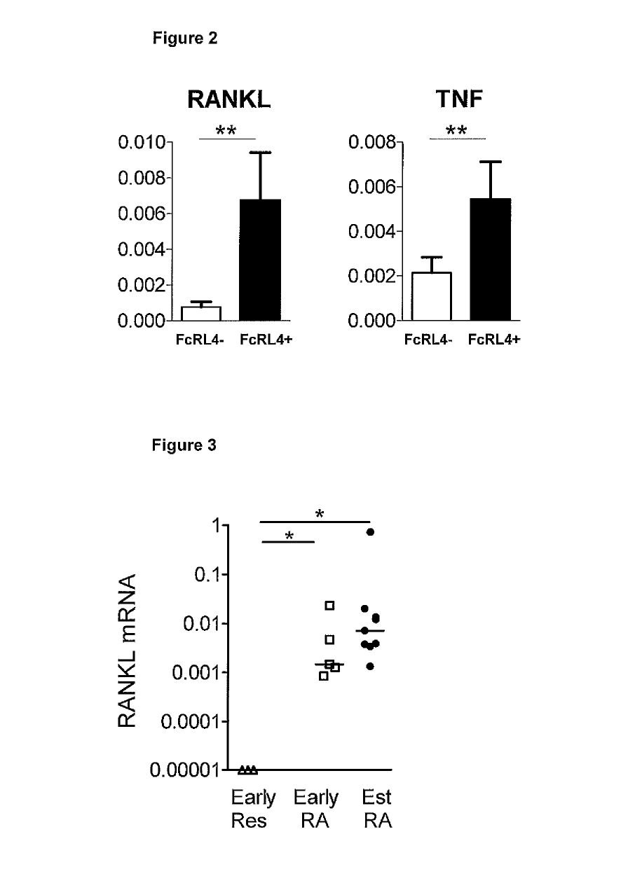

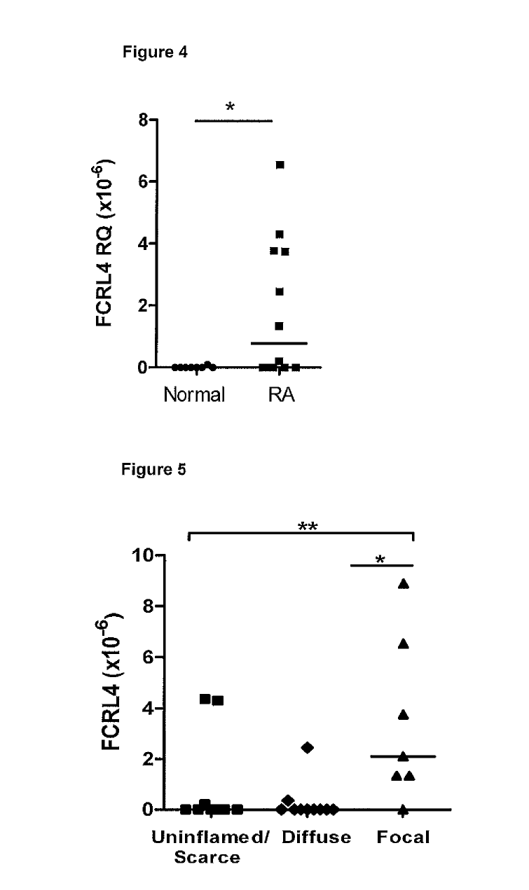

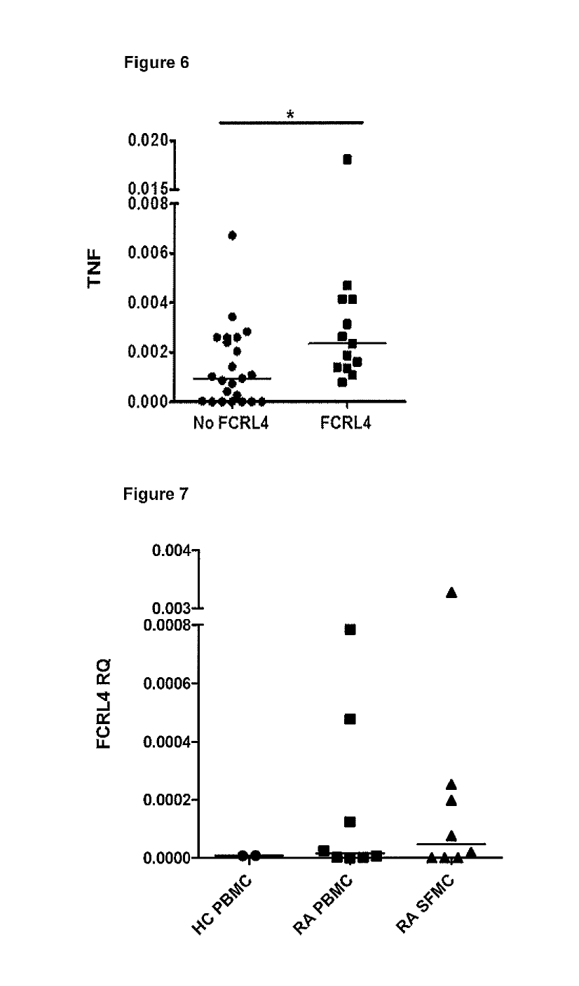

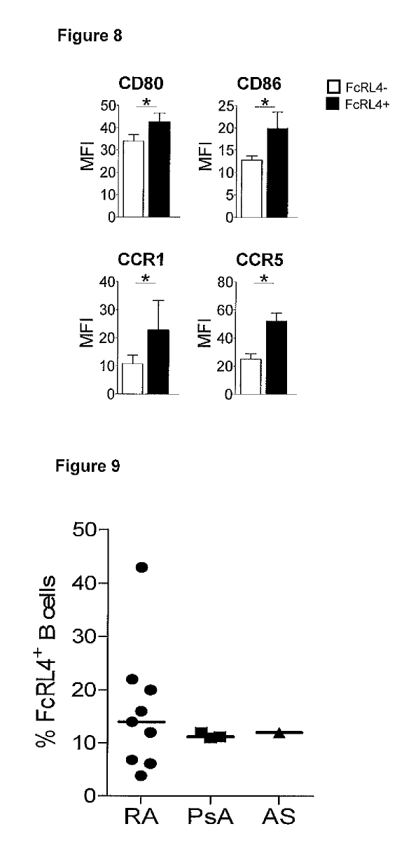

FcRL4+ cells were found at a significantly higher frequency in synovial fluid compared to peripheral blood of RA patients (FIG. 1A). FcRL4+ RANKL+ cells were detected in the synovial fluid but not peripheral blood, and RANKL+ B cells were found to be enriched in the FcRL4+ B cell population (FIG. 1B). Comparison of synovial fluid B cells selected either on the basis of RANKL or FcRL4 expression suggest that these two markers define the same B cell population due to their similarity in expression of a range of markers of B cell differentiation. The FcRL4+/RANKL+ B cells are predominantly switched memory B cells with an IgD-CD27+ or IgD-CD27− phenotype (FIG. 1C). The Wilcoxon matched pairs test was used for all comparisons; *p<0.05, **p<0.01.

FIG. 2—mRNA expression profiles for RANKL and TNF-α

mRNA levels of RANKL are higher in FcRL4+ B cells than FcRL4− B cells, and FcRL4+ B cells also express considerably higher levels of mRNA for the pro-inflammatory cytokine TNF-α.

FIG. 3—RANKL expression profile in RA patients compared to patients with resolving disease

RANKL was only found to be expressed in B cells from the synovial fluid of patients who were later diagnosed as having RA, but not in patients with resolving disease. *p<0.05, Mann-Whitney test.

FIG. 4—FcRL4 mRNA is not expressed in uninflamed synovial tissue but can be detected in synovium from patients with RA.

FcRL4 mRNA expression is significantly higher in synovial tissue from RA patients (n=12) compared to non-inflamed controls (n=8). *p<0.05, Mann-Whitney test.

FIG. 5—FcRL4 is predominantly expressed in RA synovium with focal inflammatory infiltrates

FcRL4 mRNA expression in synovial tissue from early arthritis and established RA patients is elevated in samples with focal lymphocyte aggregates compared to patients with uninflamed tissue/scarce inflammatory infiltrate, and diffuse inflammatory infiltrate patterns. *p<0.05, **p<0.01, Kruskall-Wallis test (overarching bar) and Dunn's post-test (underlying bar).

FIG. 6—TNF-α mRNA expression is higher in synovium of patients with FcRL4+ B cells

TNF-α mRNA expression is higher in matched synovial tissue samples which express FcRL4 mRNA *p<0.05, Mann-Whitney test.

FIG. 7—FcRL4 mRNA can be detected in peripheral blood samples from RA patients

FcRL4 mRNA expression can be detected in peripheral blood mononuclear cells of a proportion of RA patients by real-time PCR.

FIG. 8—FcRL4+ B cells from synovial fluid may be more effective at activating T cells.

FcRL4+ B cells have higher expression of CD80, CD86, CCR1 and CCR5 to FcRL4− B cells. *p<0.05, Wilcoxon matched pairs test; n=7.

FIG. 9—FcRL4+ B cells in rheumatoid arthritis, psoriatic arthritis and ankylosing spondylitis. The percentage of B cells expressing FcRL4 in the synovial fluid assessed by flow cytometry is comparable in rheumatoid arthritis, psoriatic arthritis (PsA) and ankylosing spondylitis (AS).

### DETAILED DESCRIPTION OF THE INVENTION

In England some 580,000 adults have RA, with around 26,000 new diagnoses each year. The National Audit Office of the UK (NAO; 2009) has estimated that RA costs the UK National Health Service around £560 million a year in healthcare costs, and that the additional cost to the economy of sick leave and work-related disability is £1.8 billion a year. Importantly, only 10% of RA patients are treated within 3 months of symptom onset. Economic modelling by the NAO suggests increasing this to 20% could result in productivity gains of £31 million for the economy over 5 years due to reduced sick leave and lost employment. On average, this could also increase quality of life by four percent over the first five years, as measured by quality adjusted life years (QALY) gained.

Early diagnosis of rheumatoid arthritis (RA) allows targeting of aggressive therapy to the patients most likely to benefit from it. About 50% of the patients attending our early arthritis clinic have spontaneously resolving disease and get better without aggressive therapy. However, there is overwhelming evidence that patients who develop RA should be treated with disease modifying drugs at the earliest possible stage. It is therefore desirable to identify markers that predict whether patients will progress to develop RA or whether their disease will be self-resolving.

In a systematic study of inflammatory cell populations purified from the rheumatoid joint, we have identified a novel pro-inflammatory population of B cells that produces the bone-destructive cytokine RANKL and other disease-relevant cytokines. Staining of RANKL in synovial biopsies from patients before and after targeted B cell therapy revealed that these RANKL-expressing cells are lost following B cell depletion.

We found that RANKL-producing B cells in the rheumatoid synovial tissue express FCRL4, a marker previously identified in B cells associated with the epithelium. (Shown in FIG. 2) We have isolated FCRL4-positive and -negative B cells from RA synovial fluid and assessed their mRNA expression, confirming that FCRL4-positive B cells express higher levels of RANKL mRNA and, interestingly also higher levels of TNF-α, a key pro-inflammatory cytokine in RA. These FCRL4-expressing cells therefore represent a novel pro-inflammatory B cell population in the joints of RA patients.

Preliminary data shown in FIG. 3 suggest that these B cells are present in synovial fluid of patients with early RA (n=3) and established RA (n=10), while they are absent from the joint of patients with spontaneously resolving disease (n=3).

In summary, we have identified a novel subset of B cells in rheumatoid arthritis characterised by expression of FcRL4 which produces RANKL and expresses high levels of TNF mRNA, indicating a destructive, pro-inflammatory role for this B cell subpopulation in RA pathogenesis. Furthermore, FcRL4/RANKL expressing B cells are a useful biomarker for progression to RA.

FCRL4+ cells are known. FcRL4 is normally expressed by a unique subset of B cells found in mucosal-associated lymphoid tissues (MALT). FcRL4+ B cells are primarily found associated with the epithelium in the tonsil and Peyer's patches, and to a lesser extent in the spleen and mesenteric lymph nodes (Falini, Tiacci et al. 2003; Poison, Zheng et al. 2006). While FcRL4+ B cells are not present in the peripheral blood of healthy individuals, FcRL4+ B cells have been described in the peripheral blood of HIV-viremic individuals (Moir, Ho et al. 2008), individuals chronically infected with P. falciparum malaria (Weiss, Crompton et al. 2009), and patients with hepatitis C virus-associated mixed cryoglobulinemia (Charles, Brunetti et al. 2011). Additionally, FcRL4+ B cells have been identified in the lymph nodes of individuals with lymphadenitis (Lazzi, Bellan et al. 2006), in the tonsils of patients with post-transplant lymphoproliferative disorders (PTLD) (Shaknovich, Basso et al. 2006), and in tumour cells of MALT lymphomas (Falini, Agostinelli et al. 2012).

Erhardt (Ehrhardt, Hijikata et al. 2008) has previously looked at the expression profile of FCRL4+ B cells from tonsils and compared the transcriptome and proteome of FcRL4+ and FcRL4− memory B cells. This comparison revealed a distinct expression profile for FcRL4+ B cells found in tonsils which included increased expression of several transcription factors, cell surface proteins, intracellular signalling molecules and regulators of the cell cycle. One of the genes Erhardt showed to be upregulated in tonsilar FcRL4+ B cells, at both the mRNA and protein level, was RANKL, the key cytokine responsible for driving bone erosion.

As we previously had made the observation that B cells in the RA synovium produce RANKL, we sought to determine whether RANKL-expressing cells we had identified as having a role in RA also belonged to the FcRL4+ B cell subset.

The Rheumatology community is increasingly embracing the concept of studying tissue from the primary site of pathology (the synovium) to predict outcome in patients with early arthritis. To this end, minimally invasive approaches to the collection of synovium such as ultrasound guided biopsy have been developed and validated and are now widely used.

Patients diagnosed as having RA are generally considered to meet or fulfil the 1987 American College of Rheumatology criteria for RA (Arnett, Edworthy et al. 1988), or alternatively the new ACR 2010 criteria (Aletaha, Neogi et al. 2010)

Preferably, patients having early arthritis have at least one clinically swollen joint. Ideally, they are seen within at least 12 weeks of symptom onset. Preferably, they have also not been treated with a DMARD or with steroids, especially in the last three months.

It is preferred that the cells are isolated from synovial fluid or peripheral blood. This may be achieved using density gradient centrifugation, for instance. The isolated cells are preferably mononuclear cells.

Surprisingly, FcRL4+ and RANKL+ B cells were not generally found within the main lymphocyte aggregates in the synovial tissue where most B cells are located; instead they were localised beneath the synovial sublining layer, or found close to the borders of B cell clusters but generally not within the lymphocyte aggregates themselves. Accordingly, the present cells are preferably sampled from beneath the synovial sublining layer and/or from around the borders of B cell clusters.

FCRL4+ cells can be identified using antibodies specific to FCRL4. These include mouse anti-FcRL4 (R&D systems) or mouse anti-FcRL4 PECy7 (Biolegend). RANKL may be identified using antibodies specific to RANKL, such as the rabbit anti-RANKL Ab (from AbCam).

Stratification of early arthritis patients may be along the following strata, preferably at 18 months:
(i) having RA according to 1987 ACR criteria;
(ii) an alternative persistent inflammatory arthritis;
(iii) an unclassified persistent arthritis; or
(iv) resolving disease—resolving arthritis (defined as the absence of clinically apparent synovial swelling at final assessment with no DMARDs or steroids having been used for the previous three months).

B cells are known to be critical importantly in the pathogenesis of RA, but so far their exact role remains poorly understood. Rituximab significantly reduces synovial inflammation and erosion by effectively removing the entire pool of circulating B cells and a large proportion of tissue-based B cells. Although Rituximab does not directly target antibody-producing plasma cells, there is a decline in the level of circulating antibodies following treatment and patients become more susceptible to infections. These problems could be solved by using therapeutic approaches that specifically target B cell populations responsible for promoting inflammation, rather than indiscriminately removing all B cells.

### FcRL4 as a Therapeutic Target

We have identified a subset of B cells not previously found at sites of chronic inflammation which is characterised by expression of FcRL4 and is capable of producing the bone-destructive and pro-inflammatory cytokines RANKL and TNF-α. FcRL4 therefore represents an attractive therapeutic target as its expression is specific for B cells and its surface expression may readily facilitate effective blocking by antibodies or small molecule inhibitors.

FcRL4 Expression by B Cells from Peripheral Blood, Synovial Fluid or Synovial Tissue of RA Patients, Provides a Useful Biomarker for Prediction of Clinical Response to Rituximab.

Currently the first line of treatment for patients failing disease-modifying anti-rheumatic drugs (DMARDs) is anti-TNF therapy, which produces a clinical response in approximately 70% of patients with established RA. Rituximab has NICE approval for RA patients refractory to anti-TNF therapy. However, it is difficult at present to predict which patients will respond better to TNF targeting and which will respond better to B cell-directed therapy. In addition, it is difficult to predict which patients will progress to severe disease and thus benefit most from aggressive therapy.

We have previously shown that after RA patients are treated with Rituximab, levels of RANKL in the synovium are significantly diminished, suggesting that one mechanism by which Rituximab exerts its clinical effects is by depleting B cells responsible for producing RANKL. Here, we show that the FcRL4+ memory B cell population in the synovium produces RANKL. Therefore, we envisage that Rituximab targets among other B cell subsets the FcRL4+ cells. We also envisage that specifically the removal of the FcRL4+ B cell subset makes a major contribution to the anti-inflammatory effect of Rituximab. Accordingly, FcRL4 expression by B cells from peripheral blood, synovial fluid or synovial tissue of RA patients, provides a useful biomarker for prediction of clinical response to Rituximab.

When a patient first presents with inflammatory arthritis, potential outcomes range from development of a persistent arthritis such as RA, to resolution of disease within a few weeks. Increasing evidence highlights the importance of the early treatment of RA to limit long-term joint damage, and the rapid identification of patients at high risk of the development of RA is thus an important objective.

### FcRL4+ or RANKL+ B Cells are Biomarkers for Progression to RA

In patients with arthritis, RANKL expression by B cells is indicative of development of RA. This is shown in Example 2 and FIG. 3, where B cells expressing RANKL mRNA were present in synovial fluid from early synovitis patients who later developed RA, but not in early synovitis patients whose disease resolved. In other words, RANKL was only found to be expressed in B cells from the synovial fluid of patients who were later diagnosed as having rheumatoid arthritis, but not in patients with resolving disease, suggesting that the FcRL4 or RANKL expressing B cells may be a biomarker for progression to RA.

Although Example 2 relates to synovial fluid, it is envisaged that, as FcRL4+ or RANKL+ B cells are also found in synovial tissue and peripheral blood and that FcRL4+ B cells and RANKL+ B cells essentially overlap, the presence of FcRL4+ or RANKL+ B cells in a sample of synovial fluid, synovial tissue and/or peripheral blood is indicative of the likelihood that the patient will develop RA.

We have also shown that FcRL4+ B cells in RA synovial fluid are switched memory B cells expressing high levels of CD20 and CD11c, and low levels of CD21. (shown in FIG. 1c) This corresponds with the phenotype of tonsillar FcRL4+ B cells in healthy individuals (Ehrhardt, Hijikata et al. 2008) and the FcRL4+ B cell population present in patients with HIV (Moir, Ho et al. 2008) and malaria (Weiss, Crompton et al. 2009).

One possible explanation for the accumulation of FcRL4+ B cells in the RA synovium may be that B cell differentiation is altered and FcRL4+ B cells, which may or may not arise independently from FcRL4− memory B cells, are induced to produce RANKL (which in addition to its role in mediating bone erosion, is also required for lymphocyte development) and other pro-inflammatory cytokines, perhaps by factors present in the local environment.

FcRL4 may therefore be useful as a predictor of response to Rituximab and as a predictor of progression to rheumatoid arthritis in patients with early undifferentiated arthritis.

### Example 1

A pro-inflammatory subset of memory B cells, FCRL4+ B cells, produces RANKL in the rheumatoid synovium

### Methods

### Patients

Synovial fluid and peripheral blood were obtained from 21 patients and synovial tissue was obtained from 4 patients fulfilling the 1987 American College of Rheumatology criteria for RA (Arnett, Edworthy et al. 1988), with a disease duration of more than 3 months. Synovial fluid was aspirated from joints under palpation or ultrasound guidance. Synovial tissue was taken by ultrasound guided biopsy and immediately frozen in liquid nitrogen. From the Birmingham early arthritis clinic, synovial fluid and peripheral blood were obtained from 6 patients with early arthritis who had at least one clinically swollen joint, were seen within 12 weeks of symptom onset, and had not been treated with a DMARD. Early arthritis patients were followed for up to 18 months then classified as having RA according to 1987 ACR criteria, an alternative persistent inflammatory arthritis, an unclassified persistent arthritis or a resolving arthritis (defined as the absence of clinically apparent synovial swelling at final assessment with no DMARDs or steroids having been used for the previous three months). All patient material was obtained in agreement with the Helsinki declaration. Local ethical approval was obtained and participants gave informed, written consent.

### Flow Cytometry and Cell Sorting

Synovial fluid was incubated with 1000 U/ml endotoxin-free hyaluronidase (Hyalase™, Wockhardt UK Ltd) at 37° C. for 15 mins to reduce viscosity. Mononuclear cells were isolated from synovial fluid and peripheral blood using density gradient centrifugation. Mononuclear cells were stained with mouse monoclonal antibodies: CD19 Pacific Blue (Biolegend), CD20 Pacific Orange (Invitrogen), CD27 APC (BD Pharmingen), IgD FITC (eBioscience), CD11c APC-Cy7 (Biolegend), RANKL PE (eBioscience), FcRL4 PECy7 (BioLegend), CD95 PerCP Cy5.5 (BD Pharmingen) and CD21 APC (eBioscience). PBS with 0.5% BSA was used as a diluent and washing buffer. Flow cytometry was performed using a Dako Cyan ADP High Performance flow cytometer (Dako). Flow cytometry data were analysed using SUMMIT™ software. For cell sorting, mononuclear cells were stained with mouse antibodies against CD19 PE (Immunotools) and FcRL4 PECy7 (Biolegend). Cells were sorted using a MoFlo cell sorter (Dako). Isolated populations used had a minimum purity of 95%.

### Immunofluorescence

Immunofluorescence staining was performed on 5 μm frozen tissue sections using mouse anti-CD20 (Dako), rabbit anti-RANKL (AbCam) and mouse anti-FcRL4 (R&D systems) or mouse anti-FcRL4 PECy7 (Biolegend). CD20 was developed with goat anti-mouse IgG2a FITC (Southern Biotech), RANKL was developed with donkey anti-rabbit Rhodamine (Jackson ImmunoResearch), and FcRL4 was developed with goat anti-mouse IgG1 Cy5 (Southern Biotech) or goat anti-mouse IgG2a Cy5 (Southern Biotech). Sections were incubated with primary antibody for 1 hour and secondary antibody for 30 mins on a shaking tablet in the dark. Sections were mounted using a DAPI-containing mounting medium.

### Taqman Low-Density Array Analysis

TaqMan low-density real-time PCR arrays (Applied Biosystems) were designed to determine expression of the following genes: IL-1α, IL-1β, IL-2, IL-4, IL-5, IL-6, IL-7, IL-10, IL-11, IL-12p35, IL-12p40, IL-13, IL-15, IL-17A, IL-18, IL-21, IL-22, IL-23p19, IL-27, TNF-α, LT-β, RANKL, APRIL, BAFF, TGF-β1, CSF-2, CSF-3, MIF, EGF, FGF2, VEGF-α, IFN-γ, IFN-α1, IFN-β, CCL2, CCL3, CCL4, CCL5, CCL11, CXCL8, CXCL12, GAPDH and 18S. RNA was extracted from sorted B cells using a Nucleospin XS kit (Machery-Nagel). A reaction mixture containing RNA, Quantitect-RT Master Mix (Qiagen) and QuantiTect Reverse Transcriptase (Qiagen) was added to a TaqMan low-density array microfluidic card. Reverse transcription and real-time PCR was performed in a 7900HT Real-Time PCR System (Applied Biosystems). Relative gene expression (RQ) was expressed as 2^{-ΔCt} where ΔCt represents the difference in Ct between 18S and the target gene.

### Results

### Identification of FcRL4+ B Cells in Synovial Fluid B Cells

To determine if FcRL4-expressing B cells were present in RA patients, synovial fluid and peripheral blood mononuclear cells from patients with established rheumatoid arthritis were stained for CD19, CD20, CD27, IgD, CD11c, RANKL, FcRL4, CD95 and CD21, and analysed using flow cytometry. We detected FcRL4 expression by CD19+ synovial fluid B cells, and this population was found at a significantly higher frequency in synovial fluid compared to peripheral blood (FIG. 1A). A population of B cells expressing both FcRL4 and RANKL was detected in the synovial fluid but not peripheral blood, and RANKL+ B cells were found to be enriched in the FcRL4+ B cell population (FIG. 1B). Comparison of FcRL4+ and FcRL4− synovial fluid B cell fractions revealed that both populations were predominantly switched memory B cells with an IgD-CD27+/−, phenotype (FIG. 1C). The FcRL4+ synovial fluid B cell population expresses considerably higher levels of CD11c than the FcRL4− B cell fraction and lower levels of CD21. Interestingly, the RANKL+ synovial fluid B cell population bears a remarkable resemblance to the FcRL4+ B cell population in terms of expression of differentiation and activation markers, being predominantly IgD-CD27+/−, expressing higher levels of FcRL4 and lower levels of CD21 than the RANKL− population.

These findings indicate that FcRL4+ and RANKL+ B cell subpopulations are broadly overlapping in terms of their cell surface marker phenotypes.

### Detection of FcRL4+ B Cells in Synovial Tissue

Synovial tissue sections from patients with established rheumatoid arthritis were stained by immunofluorescence for CD20, FcRL4 and RANKL (data not shown). FcRL4+CD20+ B cells were detected in synovial tissue, and a proportion of these FcRL4+ B cells also expressed RANKL. Morphologically, B cells expressing FcRL4 and RANKL were larger than FcRL4-negative and RANKL-negative B cells. Surprisingly, FcRL4+ and RANKL+ B cells were not found within the main lymphocyte aggregates in the synovial tissue where most B cells are located; instead they were localised beneath the synovial sublining layer, or found close to the borders of B cell clusters but only rarely within the lymphocyte aggregates themselves.

Cytokine mRNA Expression of FcRL4+ B Cells

CD19+ B cells from synovial fluid of patients with arthritis were sorted into FcRL4+ and FcRL4− fractions by FACS sorting. mRNA was extracted from sorted cells and used in a series of real-time PCR reactions in microfluidic cards to detect expression of 40 cytokines. The results indicate that mRNA levels of RANKL are significantly higher in FcRL4+ B cells than the FcRL4− fraction, and FcRL4+ B cells also express considerably higher levels of mRNA for the pro-inflammatory cytokine TNF-α (FIG. 2). This is an important observation, as TNF-α is a main driver of inflammation. It is a well validated clinical target in the treatment of rheumatoid arthritis and other autoimmune and/or chronic inflammatory conditions. Removal of a TNF-α by Rituximab can both be an explanation for the anti-inflammatory effect of Rituximab and also support the use of detection of FcRL4 expression as a biomarker to identify the patients who are most likely to respond to B cell depleting therapy. Furthermore specific, direct targeting of FcRL4 expressing B cells would allow removal of the pro-inflammatory B cells while sparing the vast majority of B cells.

### Example 2

### RANKL+ B Cell Subpopulation as a Potential Diagnostic Marker for RA

In a separate line of experiments we investigated, using similar methods to Example 1, B cells from synovial fluid from patients at a very early stage of arthritis, generally before formal diagnosis of RA. RANKL was only found to be expressed in B cells from the synovial fluid of patients who were later diagnosed as having RA, but not in patients with resolving disease (FIG. 3), suggesting that the FcRL4/RANKL expressing B cells may be a biomarker for progression to RA.

### Discussion of Examples 1 and 2

In summary, we have identified a novel subset of B cells in rheumatoid arthritis characterised by expression of FcRL4 which produces RANKL, and expresses high levels of TNF-α, indicating a destructive, pro-inflammatory role for this B cell subpopulation in RA pathogenesis. Furthermore, FcRL4/RANKL expressing B cells are a useful biomarker for progression to RA.

### Example 3

### Investigation of FCRL4 Expression as a Biomarker for the Progression of Early Arthritis to RA and Development of a Diagnostic Test that Allows Rapid Treatment Leading to Better Patient Outcome

We have collected a set of synovial biopsies from patients within the first 3 months of symptom onset. Some of these patients progress to developing RA and some have spontaneously resolving disease. As control groups, we have biopsies from non-inflamed controls and from patients with long-standing RA.

As microscopy based methods will be difficult to translate to the clinic, alternative approaches using directly translatable technology are useful. Data from gene expression databases suggest that FCRL4 expression is highly restricted to this B cell subset. A PCR-based protocol can be developed that would only require the attending clinician to transfer peripheral blood, synovial fluid or synovial needle biopsies into a commercially available container with fixative and send it to the lab for detection of FCRL4 mRNA expression using widely available generic technology.

Part 1) Assessment of the Presence of FCRL4 Expressing B Cells in the Inflamed Synovium and Testing their Association with Progression Towards RA.

Many patients have unclassified arthritis during this phase of disease. Following tissue biopsy, patients are followed for up to 18 months to determine diagnostic outcome. Biopsies investigated here will belong to 5 patient groups: 1) Early Arthritis which has progressed to RA, 2) Resolving arthritis, 3) Non-RA, persistent inflammatory arthritis (e.g. psoriatic arthritis). As controls we used samples from patients with 4) Established RA of at least 6 months' duration and 5) “normal” controls comprising patients who underwent knee arthroscopy because of unexplained knee pain who did not show inflammatory or degenerative joint pathology during arthroscopy.

### Part 2) PCR Protocol for Detection of FCRL4 Gene Expression in Clinical Samples.

We are looking to use a more easily translatable method of detection of FCRL4. While immunofluorescence staining was necessary for the intellectual framework of this project, it is a training intensive method that needs specialist equipment beyond the reach of most clinical laboratories. We therefore developed a PCR based diagnostic assay based on the detection of mRNA expression coding for FCRL4. Existing microarray data (Gene Expression Atlas) suggest that in healthy donors FCRL4 expression is limited to the FCRL4-positive B cell subset in the tonsil and to a lesser extent also in other epithelial sites such as the salivary glands. Most of the 32 different tissues tested were negative for FCRL4. We validated a protocol for detection of FCRL4 gene expression in peripheral blood, synovial fluid and biopsy material. For validation of the specificity of the PCR protocol we initially used synovial fluid cells established the sensitivity and specificity of this test for clinical samples from peripheral blood, synovial fluid and synovial biopsies.

Part 3) Quantification of FCRL4 mRNA Expression in Biopsy Material Collected from the Early Arthritis Clinic.

The above assay as shown in FIG. 7, can be used to determine FCRL4 expression in samples of synovial tissue from patients in the Birmingham Early Arthritis Cohort. One can also look at peripheral blood samples from healthy controls and newly presenting RA patients to determine whether detection of FCRL4 mRNA expression in blood is associated with RA. One can continue to develop this work in peripheral blood, synovial fluid and biopsy material from a larger patient group.

### Example 4

### Methods

### Patients

Synovial fluid, peripheral blood and synovial tissue samples were obtained from patients fulfilling the 1987 American College of Rheumatology criteria for RA, with a disease duration of more than 3 months. In addition, synovial biopsies were obtained from patients seen in the Birmingham early arthritis clinic. Early arthritis patients had at least one clinically swollen joint, were seen within 12 weeks of symptom onset, and were DMARD naïve. After 18-month follow up, patients were classified as having RA, an alternative persistent inflammatory arthritis, an unclassified persistent arthritis or a resolving arthritis (absence of synovial swelling with no DMARDs or steroids used for the previous three months).

The study was conducted in compliance with the Helsinki declaration and ethical approval was obtained from the Local Ethics Committee. All subjects gave informed, written consent. Synovial fluid was aspirated from joints under palpation or ultrasound guidance. Synovial tissue was taken by ultrasound guided biopsy and frozen immediately in liquid nitrogen.

### Flow Cytometry and Cell Sorting

Synovial fluid was incubated with 1000 U/ml endotoxin-free hyaluronidase (Hyalase™, Wockhardt UK Ltd) at 37° C. for 15 mins to reduce viscosity. Mononuclear cells were isolated from synovial fluid and peripheral blood using density gradient centrifugation. Mononuclear cells were stained with mouse monoclonal antibodies against CD19 Pacific Blue (Biolegend), CD20 Pacific Orange (Invitrogen), CD27 APC (BD Pharmingen), IgD FITC (eBioscience), CD11c APC-Cy7 (Biolegend), RANKL PE (eBioscience), FcRL4 PECy7 (Biolegend), CD95 PerCP Cy5.5 (BD Pharmingen), CD21 APC (eBioscience), CD80 PE (BD Biosciences), CD86 PerCPCy5.5 (Biolegend), CCR1 APC (R&D Systems) and CCR5 APC Cy7 (BD Biosciences). For all stainings isotype, concentration, species and label matched control antibodies were used to ensure specificity of staining. PBS with 0.5% BSA was used as a diluent and washing buffer. Flow cytometry was performed using a Dako Cyan ADP High Performance flow cytometer (Dako). Flow cytometry data were analysed using SUMMIT™ software. For cell sorting, mononuclear cells were stained with mouse antibodies against CD19 PE (Immunotools) and FcRL4 PECy7 (Biolegend). Cells were sorted using a MoFlo cell sorter (Dako). Sorted populations used had a minimum purity of 95%.

### Immunofluorescence

Immunofluorescence staining was performed on 5 μm frozen tissue sections using mouse anti-CD20 (Dako), rabbit anti-RANKL (AbCam) and mouse anti-FcRL4 PECy7 (eBioscience). CD20 was developed with goat anti-mouse IgG2a FITC (Southern Biotech), RANKL was developed with donkey anti-rabbit Rhodamine (Jackson ImmunoResearch), and FcRL4 was developed with goat anti-mouse IgG2bCy5 (Southern Biotech). Sections were incubated with primary antibody for 1 hour and secondary antibody for 30 mins on a shaking tablet in the dark. Sections were mounted using a DAPI-containing mounting medium as a nuclear counterstain. Sections were visualised using a Zeiss confocal LSM 510 microscope (Zeiss)

### Taqman Low-Density Array Analysis

TaqMan low-density real-time PCR arrays (Applied Biosystems) were designed to determine expression of the following genes: IL-1α, IL-1β, IL-2, IL-4, IL-5, IL-6, IL-7, IL-10, IL-11, IL-12p35, IL-12p40, IL-13, IL-15, IL-17A, IL-18, IL-21, IL-22, IL-23p19, IL-27, TNF-α, LT-β, RANKL, APRIL, BAFF, TGF-β1, CSF-2, CSF-3, MIF, EGF, FGF2, VEGF-α, IFN-γ, IFN-α1, IFN-β, CCL2, CCL3, CCL4, CCL5, CCL11, CXCL8, CXCL12, GAPDH and 18S. RNA was extracted from sorted B cells using a Nucleospin XS kit (Machery-Nagel). A reaction mixture containing RNA, Quantitect-RT Master Mix (Qiagen) and QuantiTect Reverse Transcriptase (Qiagen) was added to a TaqMan low-density array microfluidic card. Reverse transcription and real-time PCR was performed in a 7900HT Real-Time PCR System (Applied Biosystems). The cycling program used was 50° C. for 30 min, 94.5° C. for 15 sec then 40 cycles of 96° C. for 30 sec and 59.7° C. for 1 min. Relative gene expression (RQ) was expressed as 2^{-ΔCt} where ΔCt represents the difference in Ct between 18S and the target gene.

### Real-Time PCR

RNA was extracted using the RNeasy kit (Qiagen), reverse transcribed using the Superscript VILO kit (Invitrogen), then used in real-time PCR assays containing 12.5 ul Taqman Master Mix, 1 ul FcRL4 or 18S assay (all Applied Biosystems), 6.5 ul H₂O and 5 ul cDNA. The cycling program used was 50° C. for 2 minutes, 95° C. for 10 minutes and 40 cycles of 15 seconds at 95 and 1 minute at 60° C. Relative gene expression (RQ) was expressed as 2^{-ΔCt}.

### Results

Part 1) FcRL4 mRNA is not Expressed in Uninflamed Synovial Tissue but can be Detected in Synovium from Patients with RA.

FcRL4 mRNA expression was assessed in synovial tissue from RA patients and control subjects who were undergoing arthroscopy for treatment of non-inflammatory conditions and had no macroscopic or radiological evidence of inflammatory pathology. mRNA expression of FcRL4 was found to be significantly elevated in RA patients compared to control subjects. This indicates that FcRL4+ B cells are not present in normal synovium but develop or accumulate in the synovium during RA. This supports the concept of FcRL4 representing an attractive therapeutic target since its expression in the synovium is restricted to the disease state.

FIG. 4 shows FcRL4 mRNA expression is significantly higher in synovial tissue from RA patients (n=12) compared to non-inflamed controls (n=8).

Part 2) FcRL4 is Predominantly Expressed in RA Synovium with Focal Inflammatory Infiltrates.

Synovial tissue biopsies obtained from the early arthritis patients and established RA patients were assessed for FcRL4 mRNA expression. The histology of these tissues was scored on matching biopsies from the same joint as having an inflammatory infiltrate graded as uninflamed/scarce, diffuse (scattered inflammatory cells), or focal (containing lymphocyte aggregates). Comparison of FcRL4 expression in synovial tissue grouped by the type of infiltrate showed that FcRL4 mRNA levels were highest in tissues with focal infiltrate. As patients with focal infiltrate are likely to represent the subset of RA patients which responds to rituximab, these data indicate that use of real-time PCR to determine FcRL4 mRNA expression in synovial tissue may be important in predicting response to rituximab. FIG. 5 shows FcRL4 mRNA expression in synovial tissue from early arthritis and established RA patients is elevated in samples with focal lymphocyte aggregates compared to patients with uninflamed tissue/scarce inflammatory infiltrate, and diffuse inflammatory infiltrate patterns. *p<0.05, **p<0.01, Kruskall-Wallis test (overarching bar) and Dunn's post-test (underlying bar).

Frozen sections from synovial tissue biopsies obtained from our inception cohort comprising newly presenting, DMARD naïve patients with clinically detectable swollen joints were assessed for FcRL4 mRNA expression. The histological appearance of the inflammatory infiltrates was assessed in formaldehyde fixed paraffin embedded biopsies taken from the same joints. The type of tissue inflammation was scored as either uninflamed/scarce, diffuse, or focal by a blinded trained observer. Comparison of FcRL4 expression in synovial tissue grouped by the type of infiltrate showed that FcRL4 mRNA levels were significantly higher in tissues with a focal infiltrate, compared with diffuse or scarce infiltrates (FIG. 5). TNF-α gene expression levels were found to be significantly elevated in synovial tissue in which FcRL4 was expressed as shown in FIG. 6.

Part 3) TNF-α mRNA Expression is Higher in Synovium of Patients with FcRL4+ B Cells

FIG. 6 shows TNF-α mRNA expression is higher in matched synovial tissue samples which express FcRL4 mRNA. *p<0.05, Mann-Whitney test.

FIG. 6 shows that TNF-α mRNA expression is higher in synovial tissues from patients in the early arthritis cohort or with established RA which express FcRL4 and indicates an association between the presence of FcRL4+ B cells in the synovium and local TNF-α production. This may be relevant to the observation that FcRL4+ B cells express high levels of TNF-α mRNA and may therefore be important drivers of chronic synovial inflammation.

Part 4) FcRL4 mRNA can be Detected in Peripheral Blood Samples from RA Patients.

FIG. 7 shows FcRL4 mRNA expression can be detected in peripheral blood mononuclear cells of a proportion of RA patients by real-time PCR.

The use of real-time PCR to determine FcRL4 mRNA expression in peripheral blood shown in FIG. 7 is advantageous as it is an important method for determining response to rituximab in addition to the antibody-based methods used to detect FcRL4 protein also described herein.

Part 5) FcRL4+ B Cells from Synovial Fluid May be More Effective at Activating T Cells.

FIG. 8 shows FcRL4+ B cells have higher expression of CD80, CD86, CCR1 and CCR5 to FcRL4− B cells. *p<0.05, **p<0.01, Mann-Whitney test; n=7.

The data shown in FIG. 8 support what was previously included highlighting that FcRL4+ B cells belong to a subset that is distinct from the other B cells present in the RA synovium. Higher levels of costimulatory molecules CD80 and CD86 indicate they may be more efficient at activating T cells.

Stimulation of T cells by B cells in the synovium has been suggested to be one of the potential explanations for why removal of B cells in RA patients decreases inflammation. These data provide indirect evidence suggesting that removal of the FcRL4+ B cells may be a more efficient way of stopping T cell activation.

High levels of CCR1 and CCR5 may explain the localisation of FcRL4+ B cells in the synovium in the diseased state of RA.

Further assessment of cell surface markers using flow cytometry highlighted several phenotypic differences that distinguish FcRL4+ and FcRL4− synovial fluid B cells. FcRL4+ B cells expressed higher levels of CD95, CD11c and CD20, and lower levels of CD21 in comparison to FcRL4− B cells (FIG. 8). FcRL4+ B cells show elevated expression of the chemokine receptors CCR1 and CCR5 in comparison to FcRL4− B cells. FcRL4+ B cells were also found to express higher levels of the co-stimulatory markers CD80 and CD86 compared to FcRL4− B cells.

### Further Discussion

Remarkably we noted that FcRL4 expression was elevated in synovial tissues which had a focal inflammatory infiltrate defined by the presence of lymphocyte aggregates, indicating that FcRL4+ B cells may be associated with developing or maintaining lymphoid structures within the inflamed synovium. In keeping with this notion we have identified FcRL4+ B cells as the subset responsible for production of RANKL; RANKL is known to have an important role in lymphoid neogenesis (Kong, Yoshida et al. 1999; Knoop, Butler et al. 2011) and may therefore have a role in promoting the development of lymphocyte structures present in the rheumatoid synovium. FcRL4+ B cells were found to have elevated expression of CCR1 and CCR5 which are receptors for the T cell chemoattractants MIP-1α, MIP-1β and RANTES. It is conceivable that high expression of CCR1 and CCR5 may attract these cells to the vicinity of T cells and facilitate T cell dependent activation of FcRL4+ B cells. Altered chemokine receptor expression may account for the homing to and retention of FcRL4+ B cells in the inflamed synovium.

Current B cell depletion therapy for RA indiscriminately removes all B cells and thus has the disadvantages of reducing patients' response to infection and vaccination. We propose that in the future FcRL4 may represent a potential therapeutic target which would allow specific removal of B cells responsible for promoting inflammation, while leaving protective B cells intact.

### Example 5

### FcRL4+ B Cells are Present in Other Autoimmune and/or Chronic Conditions Affecting the Joints

### FcRL4+ B Cells are Present in Psoriatic Arthritis and Ankylosing Spondylitis.

B cells expressing FcRL4 are present in the synovial fluid of patients with psoriatic arthritis and ankylosing spondylitis at levels comparable to that seen in rheumatoid arthritis (see FIG. 9). This indicates that FcRL4+ B cells present at the site of inflammation in these diseases have a pathogenic role in promoting inflammation and T co-stimulation. Recent studies have shown that use of rituximab is effective in psoriatic arthritis and ankylosing spondylitis (Tony, Burmester et al. 2011; Jimenez-Boj, Stamm et al. 2012; Song, Heldmann et al. 2013). However, since administration of rituximab has the disadvantage of reducing B cell immunity and increasing risk of infection in patients, FcRL4 represents an alternative attractive therapeutic target by specifically removing the pathogenic B cell subpopulation.

### REFERENCES

Aletaha, D., T. Neogi, et al. (2010). “2010 Rheumatoid arthritis classification criteria: an American College of Rheumatology/European League Against Rheumatism collaborative initiative.” Arthritis and rheumatism 62(9): 2569-2581.
Arnett, F. C., S. M. Edworthy, et al. (1988). “The American Rheumatism Association 1987 revised criteria for the classification of rheumatoid arthritis.” Arthritis and rheumatism 31(3): 315-324.
Charles, E. D., C. Brunetti, et al. (2011). “Clonal B cells in patients with hepatitis C virus-associated mixed cryoglobulinemia contain an expanded anergic CD21low B-cell subset.” Blood 117(20): 5425-5437.
Edwards, J. C. and G. Cambridge (2001). “Sustained improvement in rheumatoid arthritis following a protocol designed to deplete B lymphocytes.” Rheumatology (Oxford) 40(2): 205-211.
Ehrhardt, G. R., R. S. Davis, et al. (2003). “The inhibitory potential of Fc receptor homolog 4 on memory B cells.” Proceedings of the National Academy of Sciences of the United States of America 100(23): 13489-13494.
Ehrhardt, G. R., A. Hijikata, et al. (2008). “Discriminating gene expression profiles of memory B cell subpopulations.” The Journal of experimental medicine 205(8): 1807-1817.
Falini, B., C. Agostinelli, et al. (2012). “IRTA1 is selectively expressed in nodal and extranodal marginal zone lymphomas.” Histopathology.
Falini, B., E. Tiacci, et al. (2003). “Expression of the IRTA1 receptor identifies intraepithelial and subepithelial marginal zone B cells of the mucosa-associated lymphoid tissue (MALT).” Blood 102(10): 3684-3692.
Jimenez-Boj, E., T. A. Stamm, et al. (2012). “Rituximab in psoriatic arthritis: an exploratory evaluation.” Annals of the rheumatic diseases 71(11): 1868-1871.
Kardava, L., S. Moir, et al. (2011). “Attenuation of HIV-associated human B cell exhaustion by siRNA downregulation of inhibitory receptors.” The Journal of clinical investigation 121(7): 2614-2624.
Knoop, K. A., B. R. Butler, et al. (2011). “Distinct developmental requirements for isolated lymphoid follicle formation in the small and large intestine: RANKL is essential only in the small intestine.” The American journal of pathology 179(4): 1861-1871.
Kong, Y. Y., H. Yoshida, et al. (1999). “OPGL is a key regulator of osteoclastogenesis, lymphocyte development and lymph-node organogenesis.” Nature 397(6717): 315-323.
Lazzi, S., C. Bellan, et al. (2006). “IRTA1+ monocytoid B cells in reactive lymphadenitis show a unique topographic distribution and immunophenotype and a peculiar usage and mutational pattern of IgVH genes.” The Journal of pathology 209(1): 56-66.
Moir, S., J. Ho, et al. (2008). “Evidence for HIV-associated B cell exhaustion in a dysfunctional memory B cell compartment in HIV-infected viremic individuals.” The Journal of experimental medicine 205(8): 1797-1805.
Oren, S., M. Mandelboim, et al. (2008). “Vaccination against influenza in patients with rheumatoid arthritis: the effect of rituximab on the humoral response.” Ann Rheum Dis 67(7): 937-941.
Poison, A. G., B. Zheng, et al. (2006). “Expression pattern of the human FcRH/IRTA receptors in normal tissue and in B-chronic lymphocytic leukemia.” International immunology 18(9): 1363-1373.
Shaknovich, R., K. Basso, et al. (2006). “Identification of rare Epstein-Barr virus infected memory B cells and plasma cells in non-monomorphic post-transplant lymphoproliferative disorders and the signature of viral signaling.” Haematologica 91(10): 1313-1320.
Song, I. H., F. Heldmann, et al. (2013). “One-year follow-up of ankylosing spondylitis patients responding to rituximab treatment and re-treated in case of a flare.” Annals of the rheumatic diseases 72(2): 305-306.
Thurlings, R. M., K. Vos, et al. (2008). “Synovial tissue response to rituximab: mechanism of action and identification of biomarkers of response.” Ann Rheum Dis 67(7): 917-925.
Tony, H. P., G. Burmester, et al. (2011). “Safety and clinical outcomes of rituximab therapy in patients with different autoimmune diseases: experience from a national registry (GRAID).” Arthritis Research & Therapy 13(3): R75.
Weiss, G. E., P. D. Crompton, et al. (2009). “Atypical memory B cells are greatly expanded in individuals living in a malaria-endemic area.” Journal of immunology 183(3): 2176-2182.
Yeo, L., K. M. Toeliner, et al. (2011). “Cytokine mRNA profiling identifies B cells as a major source of RANKL in rheumatoid arthritis.” Annals of the rheumatic diseases 70(11): 2022-2028.

1-20. (canceled)

21. A method for the treatment or prophylaxis of autoimmune and/or chronic disease associated with joint inflammation, comprising administering a biopharmaceutical specific for FcRL4, to thereby deplete or remove pro inflammatory (FcRL4+) B cells in the synovium or a sample.

22. The method of claim 21, wherein the autoimmune and/or chronic disease associated with joint inflammation is lupus, an arthritis or a sclerosis, or ankylosing spondylitis.

23. The method of claim 22, wherein the arthritis is psoriatic arthritis or rheumatoid arthritis.

24. The method of claim 22, wherein the biopharmaceutical specific for FcRL4 is an anti-FcRL4 antibody.

25. The method of claim 21, wherein the biopharmaceutical specific for FcRL4 is an anti-FcRL4 antibody.

26. A method of determining the likelihood of a patient developing an autoimmune and/or chronic disease associated with joint inflammation, the method comprising determining the presence of FcRL4 RNA, FcRL4-expressing B cells and/optionally RANKL-expressing B cells, in a blood sample or a synovial tissue or fluid sample, from said patient.

27. The method of claim 26, wherein the autoimmune and/or chronic disease associated with joint inflammation is rheumatoid arthritis, ankylosing spondylitis or psoriatic arthritis.

28. The method of claim 26, for distinguishing between chronic disease and resolving disease, the presence of one or more of these biomarkers being indicative of chronic disease, rather than resolving disease.

29. The method of claim 26, wherein determining the presence of FcRL4 RNA, FcRL4-expressing B cells and/optionally RANKL-expressing B cells comprises:
a. contacting the sample with a biopharmaceutical specific for FcRL4, and/or a biopharmaceutical specific for RANKL;
b. comparing the binding of the biopharmaceutical or biopharmaceuticals to a reference level; and
c. determining that the patient is likely to develop the disease based upon the increased binding of said biopharmaceutical or biopharmaceuticals.

30. The method of claim 29, wherein the biopharmaceutical specific for FcRL4 is an anti-FcRL4 antibody, and the biopharmaceutical specific for RANKL is an anti-RANKL antibody.

31. A method according to claim 26, wherein determining the presence of FcRL4 RNA, FcRL4-expressing B cells and/or RANKL-expressing B cells comprises:
a) amplifying polynucleotides encoding FcRL4;
b) the presence of said polynucleotides encoding FcRL4 being indicative that the patient is likely to develop the disease.

32. The method of claim 31, wherein the amplifying is by quantitative PCR (qPCR).

33. A method of treating a patient with an autoimmune and/or chronic disease associated with joint inflammation, comprising:
a) determining, according to a method according to claim 26, the likelihood of a patient having or developing said disease; and
b) administering a suitable treatment to a patient determined to have said condition or to a patient likely to develop said condition.

34. The method of claim 33, wherein the autoimmune and/or chronic disease associated with joint inflammation is lupus, an arthritis or a sclerosis, or ankylosing spondylitis.


SEQUENCE LISTING (2 sequences)

SEQ ID NO: 1 - protein, 515 aa, Homo sapiens
MLLWASLLAFAPVCGQSAAAHKPVISVHPPWTTFFKGERVTLTCNGFQFYATEKTTWYHRHYWGEKLTLTPGNTLEVRESGLYRCQARGSPRSNPVRLLFSSDSLILQAPYSVFEGDTLVLRCHRRRKEKLTAVKYTWNGNILSISNKSWDLLIPQASSNNNGNYRCIGYGDENDVFRSNFKIIKIQELFPHPELKATDSQPTEGNSVNLSCETQLPPERSDTPLHFNFFRDGEVILSDWSTYPELQLPTVWRENSGSYWCGAETVRGNIHKHSPSLQIHVQRIPVSGVLLETQPSGGQAVEGEMLVLVCSVAEGTGDTTFSWHREDMQESLGRKTQRSLRAELELPAIRQSHAGGYYCTADNSYGPVQSMVLNVTVRETPGNRDGLVAAGATGGLLSALLLAVALLFHCWRRRKSGVGFLGDETRLPPAPGPGESSHSICPAQVELQSLYVDVHPKKGDLVYSEIQTTQLGEEEEANTSRTLLEDKDVSVVYSEVKTQHPDNSAGKISSKDEES

SEQ ID NO: 2 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID